(19) United States
(12) Patent Application Publication
(10) Pub. No.: US 2008/0234527 A1
(43) Pub. Date: Sep. 25, 2008

(54) Method for Producing Hydrogen and System Therefor

(76) Inventors: Takaya Matsumoto, Kanagawa (JP); Toshiyuki Enomoto, Yokohama (JP); Yukinoro Kude, Yokohama (JP); Yoshinori Kobori, Yokohama (JP)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 10/592,285

(22) PCT Filed: Mar. 8, 2005
(86) PCT No.: PCT/JP2005/004397

§ 371 Date: Dec. 11, 2006

(30) Foreign Application Priority Data
Mar. 9, 2004 (JP) 2004-065894
Mar. 30, 2004 (JP) 2004-100678
Mar. 30, 2004 (JP) 2004-100688

(51) Int. Cl. C07C 4/12

(52) U.S. Cl. 585/483

(57) ABSTRACT

The present invention provides a hydrogen production method capable of producing hydrogen with good efficiency while solving problems such as separation, lower-temperature reaction and heat supply in production of hydrogen by dehydrogenation reaction of raw material oil. Within a reaction tube of a double-tube structure comprising an inner tube composed of a hydrogen separating membrane, a metallic outer tube having a plurality of internal fins, and a metal oxide layer and further a catalyst supported on the fins, hydrocarbon having cyclohexane ring is dehydrogenated to produce hydrogen and aromatic hydrocarbon, and selective membrane separating operation of hydrogen is performed within the reaction system while conducting the dehydrogenation to remove mainly the hydrogen on a permeating side and obtain mainly the aromatic hydrocarbon on a non-permeating side. The other method comprises absorbing at least part of the resulting hydrogen flow to a hydrogen absorbing (storing) alloy to make the pressure on the hydrogen permeating side of the hydrogen separating membrane lower than that on the non-permeating side.

### TECHNICAL FIELD

The present invention relates to a method and system for producing hydrogen by dehydrogenation reaction of a raw material oil composed of hydrocarbon, for example, a raw material oil mainly composed of hydrocarbon having cyclohexane ring in the field of hydrogen production.

Further, the present invention relates to a hydrogen production method, comprising making, in dehydrogenation reaction of a raw material oil composed of hydrocarbon, for example, hydrocarbon mainly having cyclohexane ring by a membrane reactor containing a hydrogen separating membrane, the pressure on the permeating side of the membrane lower than that on the non-permeating side of the membrane by using a hydrogen absorbing (storing) alloy, thereby improving the hydrogen recovery rate, and a hydrogen production system used for this method.

### BACKGROUND ART

Hydrogen is widely used in all industrial fields, including petroleum refining and chemical industry. In recent years, particularly, hydrogen energy has increasingly attracted attention as a future energy, and studies have been made focusing around a fuel cell. However, since hydrogen gas has a large volume per calorie and also needs a large energy for liquefaction, the system for storage and transport of hydrogen is an important problem. Further, development of new infrastructure for hydrogen supply is also needed (Refer to Quarterly IAE Review, by Nori KOBAYASHI, Vol. 25, No. 4, pp. 73-87 (2003)).

On the other hand, since liquid hydrocarbon has an advantage that existing infrastructures can be used, in addition to easiness of handling with a large energy density, compared with hydrogen gas, processes for hydrocarbon storage and transportation, and hydrogen production therefrom on demand are important.

The production of hydrogen has been extensively performed by known techniques such as steam reforming of methane or light paraffin, self-thermal reforming, and partial oxidation. However, these reactions require high temperature. Further, when intended for on-site power generation by fuel cell, particularly, solid polymer electrolytic fuel cell, a shift reactor and a carbon monoxide remover by CO selective oxidation or methanation are required in the latter stage thereof, resulting in an extremely complicated process. When intended for a hydrogen station for automobile, hydrogen must be made to high purity hydrogen by use of PSA (pressure swing adsorption). The same goes for a reforming system of methanol, which requires a remover of carbon monoxide for the on-site case, and the PSA for the hydrogen station.

On the other hand, production of hydrogen by dehydrogenation of liquid hydrocarbon has a simplified production process since the reaction is simple. Further, since the products thereof are hydrogen that is gas and unsaturated hydrocarbon that is liquid in ordinary temperature, this method has the feature that the both can be relatively easily separated. Particularly, it is suitable for small-scaled hydrogen production to use hydrocarbon having cyclohexane ring as raw material and dehydrogenate the cyclohexane ring to aromatic ring, because the reaction easily proceeds in the presence of a dehydrogenating catalyst, and separation of hydrogen and aromatic hydrocarbon that are products is relatively easy (refer to Engineering Materials by Masaru ICHIKAWA, Vol. 51, No. 4, pp. 62-69 (2003)).

However, although most of aromatic hydrocarbon can be liquefied and separated from hydrogen by reducing the temperature of the produced hydrogen and aromatic hydrocarbon to room temperature in the atmospheric pressure, the aromatic hydrocarbon is included in hydrogen gas in a quantity according to vapor pressure at room temperature. In the case of toluene, for example, contamination thereof at 15° C. in the atmospheric pressure is about 2.1%. Accordingly, in a case needing an increased purity of hydrogen such as fuel cell application, the separation of hydrogen from aromatic hydrocarbon becomes an important subject.

As the separation method, separation by cooling requires a low temperature of about −30° C. at ordinary pressure for attaining a hydrogen concentration of not less than 99.9%. Cooling to −30° C. using a freezer is not a preferable removing method because energy efficiency therefor is made low and a large facility is required in hydrogen production.

Further, adsorptive separation by adsorption to an adsorbent for separation requires desorption and recovery of aromatic hydrocarbon from the adsorbent after adsorption and regeneration of the adsorbent. Particularly, PSA (pressure swing adsorption) for performing adsorption and desorption by varying pressure is well known, but this has disadvantages that the recovery rate of hydrogen gas and the entire efficiency are low, and operations such as pressure rising and pressure reducing are needed, thereby resulting in an enlarged facility.

As a separation method other than the above, membrane separation has the feature of good energy efficiency, and palladium membrane, polymer membrane, ceramic membrane, and carbon membrane are mainly adapted as the separating membrane therefor. In purification of hydrogen, the palladium membrane has been put into practical use for the purpose of high purity hydrogen purification (refer to Membrane Treatment Technique System (First Vol.) edited by Masayuki NAKAGAKI, Fujitec Corp., pp. 661-662 and pp. 922-925 (1991)).

In the membrane separation, since the non-permeating side of the membrane must be raised in pressure, hydrogen generation reaction (dehydrogenation reaction) must be performed at an increased pressure, or the pressure of generated gas after reaction must be increased. The increase in pressure of generated gas lowers energy efficiency in hydrogen production. In the dehydrogenation reaction, if the reaction pressure is raised, the reaction temperature must be increased because of limitations by chemical equilibration.

However, the dehydrogenation reaction of hydrocarbon mainly having cyclohexane ring must be performed at a lower temperature in order to suppress decomposition reaction that is a side reaction. For example, dehydrogenation reaction of methylcyclohexane must be performed at a temperature of not higher than 360° C. It was difficult to reduce the temperature of this process due to limitations of equilibrium. To solve this problem, as a technique for attaining improvement in hydrogen yield and a lower-temperature reaction by selectively removing hydrogen produced in the dehydrogenation process from a reaction field by use of a membrane reactor incorporated with a hydrogen separating membrane, for example, there is disclosed a technique for performing dehydrogenation reaction of cyclohexane by using a porous ceramic membrane which hydrogen selectively permeates, in Japanese Patent Application Laid-Open No. 4-71638. Further, there are also disclosed hydrogen production techniques by reactive separation using palladium membrane in Japanese Patent Application Laid-Open Nos. 3-217227 and 5-317708, respectively.

However, each of these techniques uses an inert gas such as argon as sweep gas, which is not practicable from the point of the purity of resulting hydrogen. The dehydrogenation of cyclohexane ring is a large endothermic reaction. For example, the reaction enthalpy in dehydrogenation of cyclohexane is 50 kcal/mol, and the reaction enthalpy per cyclohexane ring is substantially equal thereto even in a hydrocarbon with substituent. Since heat transfer is rate-limited in a general solid catalyst (particle, pellet, extrusion molded body, etc.) having an active metal supported by an oxide, the temperature of catalyst is reduced due to insufficient heat supply to catalyst from out of the reactor, resulting in reduction of reaction efficiency. In reactive separation, particularly, heat supply is disadvantaged by just the increase in volume of the hydrogen separating membrane which does not take part in the reaction, from the point of the relation of heat transfer area/catalyst quantity. This tendency is more remarkable as LHSV is larger. In relation to this, there is disclosed a steam reforming method of methanol comprising using a heat conductive catalyst having an ultra-fine grain catalytic material supported on the surface of a continuous metal base in Japanese Patent Application Laid-Open No. 5-116901, which includes the description that hydrogen and carbon monoxide can be obtained in high yield.

However, the reforming reaction of methanol has the disadvantage that too many processes are required as a small-scaled hydrogen production method as described above.

### DISCLOSURE OF INVENTION

It is an object of the present invention in a first aspect to provide a hydrogen production method and a hydrogen production system capable of producing hydrogen with good efficiency while solving the problems in the production of hydrogen by dehydrogenation of a raw material oil composed of hydrocarbon, for example, a raw material oil mainly composed of hydrocarbon having cyclohexane ring, such as separation, lower-temperature reaction, and heat supply.

As the earnest studies to solve the above problems, the present inventors found out a method capable of efficiently producing hydrogen by using, in production of hydrogen by dehydrogenation of hydrocarbon, a membrane reactor capable of selectively removing hydrogen, which comprises an active metal-supported catalyst set on a metal oxide layer on the surface of a heat conductive support, and optimizing the reaction condition thereof.

Further, the present inventors found out, as a second aspect, a method capable of efficiently producing hydrogen by using a membrane reactor capable of selectively removing hydrogen in production of hydrogen by dehydrogenation of hydrocarbon, and further connecting a hydrogen absorbing (storing) alloy to the permeating side thereof to make the pressure of the permeating side lower than that of the non-permeating side, thereby improving hydrogen recovery rate.

Namely, firstly, a method for producing hydrogen according to the present invention in the first aspect comprises providing a hydrogen removing means using a hydrogen separating membrane within a dehydrogenation reaction system adapted to dehydrogenate hydrocarbon having cyclohexane ring in a flow type reaction tube containing a catalyst supported by a carrier composed of a metallic heat conductive support having a metal oxide layer localized on the surface thereof to produce hydrogen and aromatic carbon; and performing membrane separating operation through the hydrogen separating membrane while conducting the dehydrogenation reaction to remove mainly hydrogen to the permeating side thereof and obtain mainly aromatic hydrocarbon on the non-permeating side thereof.

The catalyst facilitates heat adjustment for dehydrogenation because the metal support is used therefor, and the device can be simplified because dehydrogenation and hydrogen separation are simultaneously performed.

Secondarily, the present invention in the first aspect involves that, in the method for producing hydrogen of the first mode described above, the hydrocarbon having cyclohexane ring includes methylcyclohexane, and toluene produced by dehydrogenation thereof is separated.

According to the membrane separation of the present application, separation of toluene can be also easily performed.

Thirdly, the present invention in the first aspect involves that, in the method for producing hydrogen of the first and second modes described above, the hydrogen separating membrane is a ceramic membrane.

According to the ceramic membrane separation, particularly, separation of hydrocarbon is facilitated.

Fourthly, the present invention in the first aspect involves that, in the method for producing hydrogen of the first to third modes described above, the hydrogen separating membrane is a metallic membrane containing 100-10 mass % of Pd.

According to the separation using such a metallic membrane, hydrocarbon can be easily separated.

Fifthly, the present invention in the first aspect involves that, in the method for producing hydrogen of the first to fourth modes described above, the carrier in the catalyst is a carrier containing alumina.

This carrier is advantageous for heat conduction.

Sixthly, the present invention in the first aspect involves that, in the method for producing hydrogen of the first to the fifth modes described above, the reaction tube has a double-pipe structure with an outer tube composed of the metallic heat conductive support and an inner tube composed of the hydrogen separating membrane, a plurality of metallic heat conductive fin-like projections extending inwardly from the outer tube are provided long in a flowing direction in the clearance of the double tube, and the metal oxide layer is localized on at least the fin-like projection surface to support the catalyst.

According to the double tube with internal fin, excellent heat efficiency of dehydrogenation can be provided, and simultaneous execution of dehydrogenation and membrane separation can be conveniently performed.

Seventhly, the present invention in the first aspect involves a hydrogen production reaction tube for simultaneously performing dehydrogenation of hydrocarbon and membrane separation of resulting hydrogen, which is a double-tube flow type reaction tube with an outer tube composed of a metallic heat conductive support and an inner tube composed of a hydrogen separating membrane, comprising a plurality of metallic heat conductive fin-like projections and extending inwardly from the outer tube, which are provided long in a flowing direction in the clearance of the double tube, and a metal oxide layer localized on at least the fin-like projection surface to support a catalyst.

This double-tube membrane reactor is advantageously adaptable to the present application.

Eighthly, the present invention in the first aspect involves a method for producing hydrogen, comprising providing a hydrogen removing means using a hydrogen separating membrane within a reaction system for reacting hydrocarbon in a flow type reaction tube to produce hydrogen and a reaction product; and performing membrane separating operation through the hydrogen separating membrane while conducting the dehydrogenation reaction to remove mainly hydrogen on the permeating side of the membrane and obtain mainly a reaction product on the non-permeating side of the membrane, wherein the hydrogen partial pressure on the permeating side of the membrane is reduced by carrying steam to the permeating side.

The use of steam for reducing the hydrogen partial pressure facilitates the subsequent treatment.

Ninthly, the present invention in the first aspect involves a method for producing hydrogen, comprising: providing a hydrogen removing means using a hydrogen separating membrane within a reaction system for reacting hydrocarbon on a catalyst layer in a flow type reaction tube to produce hydrogen and a reaction product; and performing membrane separating operation through the hydrogen separating membrane while conducting the dehydrogenation reaction to remove mainly hydrogen on the permeating side of the membrane and obtain mainly the reaction product on the non-permeating side of the membrane, wherein a hydrogen recovery rate of not less than 80% is obtained by producing the hydrogen with a reaction pressure of not less than 0.4 MPa by absolute pressure, a permeating-side pressure of the hydrogen separating membrane of not more than 0.12 MPa by absolute pressure, and a catalyst layer outlet temperature ranging from not lower than 300° C. to not higher than 360° C.

The adaptation of the above-mentioned reaction condition in combination with the use of the so-called membrane reactor can ensure the hydrogen recovery rate of not less than 80%.

Tenthly, the present invention in the first aspect involves a method for producing hydrogen, comprising: providing a hydrogen removing means using a hydrogen separating membrane within a reaction system for reacting hydrocarbon on a catalyst layer in a flow type reaction tube to produce hydrogen and a reaction product; and performing membrane separating operation through the hydrogen separating membrane while conducting the dehydrogenation reaction to remove mainly hydrogen on the permeating side of the membrane and obtain mainly the reaction product on the non-permeating side of the membrane, wherein a hydrogen recovery rate of not less than 80% is obtained by producing the hydrogen with a reaction pressure of not less than 0.4 MPa by absolute pressure, a permeating-side pressure of the hydrogen separating membrane of not more than 0.12 MPa by absolute pressure, a permeating-side outlet hydrogen partial pressure of not more than 0.05 MPa by absolute pressure by carrying steam to the permeating side of the hydrogen separating membrane, and a catalyst layer outlet temperature ranging from not lower than 270° C. to not higher than 360° C.

The adaptation of the above-mentioned reaction condition in combination with the use of the so-called membrane reactor can ensure the hydrogen recovery rate of not less than 80%.

Eleventhly, the present invention in the first aspect involves a method for producing hydrogen, comprising: providing a hydrogen removing means using a hydrogen separating membrane within a reaction system for reacting hydrocarbon on a catalyst layer in a flow type reaction tube to produce hydrogen and a reaction product; and performing membrane separating operation through the hydrogen separating membrane while conducting the dehydrogenation reaction to remove mainly hydrogen on the permeating side of the membrane and obtain mainly the reaction product on the non-permeating side of the membrane, wherein a hydrogen recovery rate of not less than 80% is obtained by producing the hydrogen with a reaction pressure of not less than 0.2 MPa by absolute pressure, a permeating-side pressure of the hydrogen separating membrane of not more than 0.12 MPa by absolute pressure, a permeating-side outlet hydrogen partial pressure of not more than 0.01 MPa by absolute pressure by carrying steam to the permeating-side of the hydrogen separating membrane, and a catalyst layer outlet temperature ranging from not lower than 220° C. to not higher than 360° C.

The adaptation of the above-mentioned reaction condition in combination with the use of the so-called membrane reactor can ensure the hydrogen recovery rate of not less than 80%.

The present invention in the second aspect, firstly, involves a method for producing hydrogen, comprising: continuously permeating and separating, within a flow type reaction system provided with a dehydrogenation catalyst and a hydrogen separating membrane, produced hydrogen through the hydrogen separating membrane while dehydrogenating hydrocarbon; and absorbing at least part of the resulting hydrogen flow to a hydrogen absorbing (storing) alloy to make the pressure on the hydrogen permeating side of the hydrogen separating membrane lower than that on the non-permeating side thereof.

Secondarily, the present invention in the second aspect involves that, in the method for producing hydrogen of the above-mentioned first mode in the second aspect, the hydrocarbon includes hydrocarbon having cyclohexane ring.

Thirdly, the present invention in the second aspect involves that, in the method for producing hydrogen of the above-mentioned second mode in the second aspect, the hydrocarbon having cyclohexane ring is methylcyclohexane.

Fourthly, the present invention in the second aspect involves that, in the method for producing hydrogen of the above-mentioned first to third modes in the second aspect, the hydrogen separating membrane is a ceramic membrane.

Fifthly, the present invention in the second aspect involves that, in the method for producing hydrogen of the above-mentioned first to fourth modes in the second aspect, the hydrogen separating membrane is a metallic membrane containing 100 to 10 mass % of Pd.

Sixthly, the present invention in the second aspect involves a hydrogen production system for executing the methods for producing hydrogen of the first to fifth modes in the second aspect of the present invention, comprising two or more flow type membrane reactors, two or more hydrogen absorbing (storing) alloy units, a cooler, passages for connecting them, and a passage switching means, and being adapted to perform absorption and desorption of hydrogen in the hydrogen absorbing (storing) alloy units by periodically switching the passages.

Seventhly, the present invention in the second aspect involves that, in the hydrogen production system of the above-mentioned sixth mode in the second aspect, comprising two or more flow type membrane reactors, two or more hydrogen absorbing units, a cooler, passages for connecting them, and a passage switching means, the passage switching means periodically switches a flow for cooling a permeated hydrogen flow from one flow type membrane reactor by the cooler to absorb it to one hydrogen absorbing (storing) alloy unit and a flow for supplying a permeated hydrogen flow from the other flow type membrane reactor to the hydrogen absorbing (storing) alloy unit which absorbed the hydrogen without passing through the cooler, whereby hydrogen from the flow type membrane reactor and desorbed hydrogen from the hydrogen absorbing (storing) alloy are obtained.

The effect of the invention in the first aspect is as follows. Namely, hydrogen can be efficiently produced in the range of optimized reaction conditions according to the method of the present invention characterized by setting an active metal-supported catalyst on a metal oxide layer on a heat conductive support surface, in production of hydrogen by dehydrogenation reaction from hydrocarbon mainly having cyclohexane ring, and using a membrane reactor capable of selectively removing hydrogen.

### THE EFFECT OF THE INVENTION

The effect of the invention in the second aspect is as follows. Namely, hydrogen can be efficiently produced according to the method of the present invention characterized by using a membrane reactor capable of selectively removing hydrogen, in production of hydrogen by dehydrogenation reaction from hydrocarbon mainly having cyclohexane ring, and further connecting hydrogen absorbing (storing) alloy to the permeating side thereof to reduce the pressure on the permeating side.

### BRIEF DESCRIPTION OF DRAWINGS

FIG. 1 is a perspective view of an internal fin type membrane reactor in the first aspect of the present invention with a cross-sectional view thereof on the left side, in each of which drawings an inner tube is not shown;

FIG. 2 is a conceptual diagram of a hydrogen production system according to the present invention in the first aspect;

FIG. 3 is a schematic sectional view of a membrane reactor comprising catalyst particles filled in the clearance of a double-tube structure having an inner tube composed of a hydrogen separating membrane, which was used in Example 1 in the first aspect, and also used in Examples 1 and 2 and Comparative Example in the second aspect;

FIGS. 4(a), (b) and (c) are conceptual diagrams of one embodiment of a hydrogen production system according to the present invention in the second aspect;

FIGS. 5(a), (b) and (c) are conceptual diagrams of another embodiment of the hydrogen production system according to the present invention in the second aspect; and

FIG. 6 is a conceptual diagram of the operation in Examples 1 and 2 in the second aspect.

### BEST MODE FOR CARRYING OUT THE INVENTION

The first aspect will be described first in detail, and the second aspect will be then described.

In the present invention, a so-called membrane reactor is used. More accurately, the membrane reactor referred to in the present invention is a flow type reaction tube comprising a hydrogen removing means using a hydrogen separating membrane within the reaction tube. Generally, the reactor frequently has a double-tube structure as the whole, with the hydrogen separating membrane constituting an inner tube of the reaction tube.

Production of hydrogen by dehydrogenation using such a reaction tube is performed as follows.

Namely, a substrate is fed from one side of a flow type reaction tube with the clearance of the double tube as a reaction field of dehydrogenation, and dehydrogenated with a catalyst present in the clearance to produce hydrogen and reaction product, and the produced hydrogen is simultaneously membrane-separated in situ through the hydrogen separating membrane constituting the inner tube, selectively permeated into the inner tube of the double tube, and then discharged out of the system, whereby high purity hydrogen is obtained.

According to this, the separation of hydrogen can rapidly performed, and the dehydrogenation reaction easily proceeds because heat can be easily supplied to the double tube clearance where the dehydrogenation reaction that is an endothermic reaction is executed by a proper heating means from the outside of the tube. As the proper heating means from the outside of the tube, a known method such as heating by a heating medium can be properly adapted.

As the hydrogen separating membrane, a metallic membrane, a porous inorganic membrane or the like, which can selectively separate hydrogen from a mixed gas of hydrocarbon and hydrogen, is preferably used. The metallic membrane as the hydrogen separating membrane comprises a metal thin film formed on the inner surface or outer surface of a tubular porous metal support having pores or a tubular porous ceramic support having pores, and as the metal thin film, a metal film containing 100-10 mass % of Pd, or a metal film containing 80-10 mass % of at least one metal selected from Ag, Cu, V, Nb, and Ta is preferable.

An optional method can be selected to form the metal thin film, concretely including electroless plating, vacuum deposition, rolling and the like. The porous inorganic film as the hydrogen separating membrane comprises a ceramic thin film controlled in pore diameter, formed on the inner surface or outer surface of a tubular porous ceramic support having pores. Since the porous inorganic film performs selective separation by molecular sieve effect, the pore size of the thin film part is set to preferably from not less than 0.3 nm to not more than 0.7 nm, further preferably to from not less than 0.3 nm to not more than 0.5 nm. As the material of the ceramic film, known ceramic materials are usable, and silica, alumina, titania, glass, silicon carbide, and silicon nitride are particularly preferred.

The catalytically active main component in the dehydrogenation catalyst of the present invention is a component having dehydrogenating activity, which can be optionally selected. However, elements of the groups VII, VIII and IB of the periodic table, concretely, iron, cobalt, nickel, copper, ruthenium, rhodium, palladium, silver, rhenium, osmium, iridium, platinum, and gold are preferably used. Among them, nickel, palladium, platinum and rhenium are further preferable. These elements may be used in combination of two or more thereof. The method for including such a catalytically active main component in a formed catalyst can be optionally selected, but impregnation is preferably adapted, concretely including Incipient Wetness method, evaporation to dryness, and the like. Compounds of these elements are preferably water-soluble salts, and preferably impregnated as aqueous solutions. The water-soluble compounds preferably include chlorides, nitrates, and carbonates.

An additive may be made coexistent in the catalyst as occasion demands. Preferred additives include a basic material. The coexistence of the basic material enables suppression of a side reaction such as decomposition resulted from acidity and suppression of deterioration of catalyst by carbonaceous deposition. Although the kind of basic materials is optionally selected, compounds of elements of the groups IA and IIA are preferred. Concretely, compounds of lithium, sodium, potassium, rubidium, cesium, beryllium, magnesium, calcium, strontium, and barium are preferably used. These compounds are preferably water-soluble materials and, further preferably, chlorides, sulfates, nitrates and carbonates. The content of the basic material is preferably set in the range between 0.1 and 100 by weight ratio to the catalytically active main component. The method for including such a basic material in a catalyst body can be optionally selected, but impregnation is preferably adapted, concretely, including Incipient Wetness Method, evaporation to dryness Method and the like.

A catalyst is used for dehydrogenation. As the catalyst of the present invention, a solid catalyst having dehydrogenating activity is preferably used to execute the reaction. As the solid catalyst, a catalyst having the catalytically active main component supported by a carrier can be suitably used.

Known catalysts supported by granular and pellet-shaped carriers can be also used. The conventional granular and pellet-shaped catalysts can be used in the manner of being simply filled in the double tube clearance, for example.

As the carrier, a stable metal oxide is preferably used from the point of that it has high mechanical strength, thermal stability and a large surface area. Further preferably, the metal oxide is formed on the surface of a support with high heat conductivity (heat conductive support).

Concrete examples of the metal oxide as the carrier include alumina, silica, titania, zirconia, and silica alumina. Among them, alumina, silica and a mixture thereof are further preferable.

The heat conductive support is defined, in the present invention as the first aspect, as a catalyst body based on a material having a heat conductivity at 300 K of not less than 10 W/m·K. The base body of this heat conductive catalyst body is preferably a metal, including those having an oxide film or the like on the surface. Concretely, generally used metals and alloys can be optionally used as the metal of the base body and, particularly, aluminum or metals and alloys having aluminum on the surface are preferably used.

The adaptation of metal as the base body has the effect of enhancing the heat conductivity of the catalyst body to hasten heat supply, resulting in improvement in reaction efficiency. Namely, since the dehydrogenation reaction is an endothermic reaction, heating the dehydrogenation reaction is facilitated by using a metal tube as the metal base body in the double tube itself as described above, and supplying heat by a proper heating means from the outside of the double tube to impart heat to the dehydrogenation reaction within the clearance of the double tube.

Further, current is directly carried by using the conductivity as the metal of the catalyst body, whereby, for example, the reaction tube can be rapidly heated to remarkably shorten the start-up time of the reactor.

The surface of the metal base body is preferably treated so as to have a large surface area in order to impart the function as the carrier of the catalytically active main component. Although known methods are adaptable for this treatment, it is preferable to increase the surface area based on anodic oxidation, for example, as described in Japanese Patent Application Laid-Open No. 2002-119856. It is preferable to form a stable oxide layer with high surface area of metal such as alumina on a base body surface, for example, a base body surface increased in surface area. This metal oxide layer can be formed, for example, by applying and drying an alumina hydrate sol to the base body surface increased in surface area followed by baking.

The shape of the catalyst carrier, including the metal base body, can be optionally determined, including a sheet-like, tubular, mesh-like or honeycomb-like shape or a fin-like projection which is directly set within the reaction tube.

Since the reaction in the double tube clearance is dehydrogenation reaction that is an endothermic reaction, the catalyst present in the clearance preferably makes direct contact with a heat supply source for efficiently performing the supply of dehydrogenation heat. Therefore, a form as a heat exchanger is preferably adapted, wherein the area to make contact with the substrate is increased by making the catalyst carrier into a sheet-like, tubular or internal fin shape which makes direct contact with the outer tube of the double tube. Any shape that can directly make contact with the heating source and increase the contact area with the substrate can be adapted. For example, as an internal fin type, a plurality of fin-like projections extending from the outer tube of the double tube structure toward the inner tube are provided long in the flowing direction of substrate with the outer tube of the double tube structure as a part of the metal base body, and a metal oxide layer is formed on the fin surface so that the catalyst can be supported. This metal base body can be composed of a metal such as aluminum, and increased in surface area by the method described above. The catalyst can be properly supported also on a portion other than the fins such as the inner surface of the outer tube by the above-mentioned method. Since the inner tube can be supported separately, a plurality of fins extending from the outer tube to the inner tube need not be in contact with the hydrogen separating membrane constituting the inner tube.

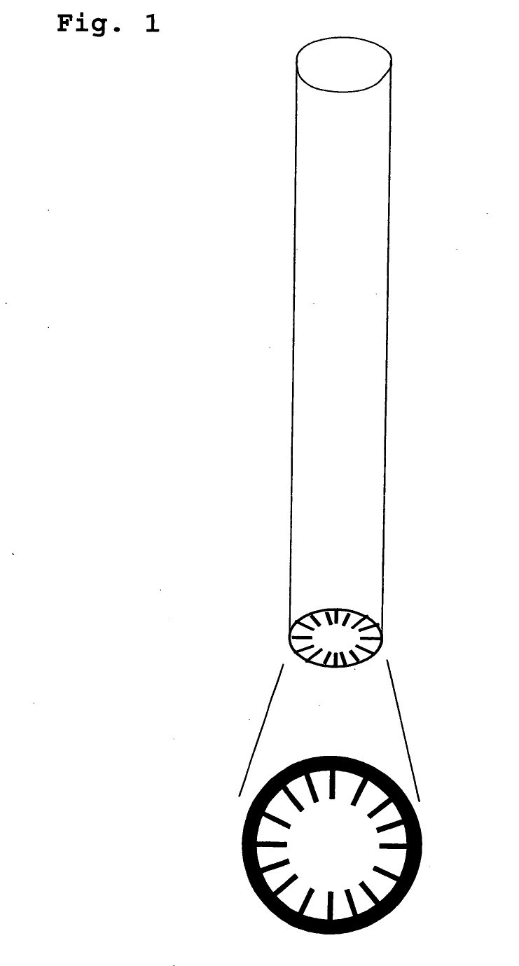

For example, an internal fin type reactor concretely shown in FIG. 1 is adaptable. FIG. 1 shows an outer tube of a double tube, which is used for dehydrogenation reaction with an inner tube consisting of a hydrogen separating membrane, not shown, being inserted to the inside thereof. The plurality of fins need not be in contact with the hydrogen separating membrane of the inner tube. The number, height, thickness or the like of the fins can be properly selected as long as it is not limited from the point of strength or the like. The fin shape can be set to a proper shape having an opening for increasing the surface area, although a sheet-like shape extending directly vertically from the outer tube surface is shown in FIG. 1.

Since such an internal fin type can make direct contact with a heating source arranged out of the tube, and has an increased contact area with the substrate passing in the clearance, heating is preferably facilitated.

The catalyst is not necessarily arranged in contact with the hydrogen separating membrane, and what is of paramount importance is that hydrogen produced by the effect of catalyst is immediately and simultaneously subjected to hydrogen membrane separating operation in situ and selectively discharged out of the dehydrogenation reaction system.

The raw material of the dehydrogenation in the present invention is preferably hydrocarbon, further preferably, hydrocarbon having cyclohexane ring. Concrete examples thereof include cyclohexane and alkyl-substituted derivatives of cyclohexane, decalin and alkyl-substituted derivatives of decalin, and tetralin and alkyl-substituted derivatives of tetralin. Most preferable are methylcyclohexane, dimethylcyclohexanes, decalin, and methyl decalins. Such a hydrocarbon having cyclohexane ring may be a mixture of two or more hydrocarbons. Further, other compounds, for example, hydrocarbon having no cyclohexane ring, may be properly contained as long as they do not bring about obstacles to the reaction.

When the hydrocarbon having cyclohexane ring is used as the raw material, products of dehydrogenation are hydrogen and unsaturated hydrocarbon, which is mainly composed of aromatic hydrocarbon. These can be returned to the raw material hydrocarbon by recovering and hydrogenating. Otherwise, they can be used also as the fuel of the heat source necessary for the dehydrogenation reaction as occasion demands. Since the aromatic hydrocarbon generally has a high octane value, a material with suitable boiling point can be used as gasoline substrate. Otherwise, it can be used also as a chemical product.

The reaction condition is properly selected according to the kind of raw material and the kind of reaction. The reaction pressure is set preferably to from not less than 0.1 MPa to not more than 2.0 MPa, further preferably to from not less than 0.1 MPa to not more than 1.0 MPa. In the specification, the pressure is shown by absolute pressure unless particularly referred to. Although a higher reaction temperature is preferable from the point of chemical equilibrium, a lower temperature is preferable from the point of energy efficiency. The reaction temperature is preferably from not lower than 200° C. to not higher than 400° C., more preferably from not lower than 220° C. to not higher than 360° C., and most preferably from not lower than 270° C. to not higher than 360° C. Hydrogen may be added to the raw material for the purpose of preventing deactivation of catalyst or from a reason in operation of the device although it is disadvantageous from the point of chemical equilibrium. When hydrogen is added to the raw material, the ratio of hydrogen to raw material is preferably set to from not less than 0.01 to not more than 1 by mole ratio.

The preferable range of LHSV (liquid hourly space velocity) is generally from not less than 0.2 v/v/hr to not more than 20 v/v/hr, although it is varied depending on the activity of catalyst.

The hydrogen produced by dehydrogenation reaction, which is in a mixed state with reaction product such as aromatic hydrocarbon, is immediately subjected in situ to the membrane separating operation using a hydrogen separating membrane in the present invention. When the inner tube of the double tube is constituted by the hydrogen separating membrane, the operation is performed with the innermost side of the double tube as the hydrogen permeating side and with the clearance of the double tube as the non-permeating side.

The permeating-side pressure of the hydrogen separating membrane in the membrane separation reactor is set preferably to not more than 0.2 MPa, further preferably to not more than 0.12 MPa. Further, it is preferable to supply an inert gas to the permeating side of the hydrogen separating membrane for the purpose of reducing the permeating-side hydrogen partial pressure. The permeating-side hydrogen partial pressure is set preferably to not more than 0.1 MPa, more preferably to not more than 0.05 MPa. As the inert gas, steam which is easily separable by condensation is preferably adapted. When application to solid polymer electrolytic type fuel cell is intended, steam is further preferable for this purpose from the point that steam can be introduced without removal. By adding steam to the permeating side, high purity hydrogen can be produced even with a permeating-side hydrogen partial pressure of not more than 0.05 MPa.

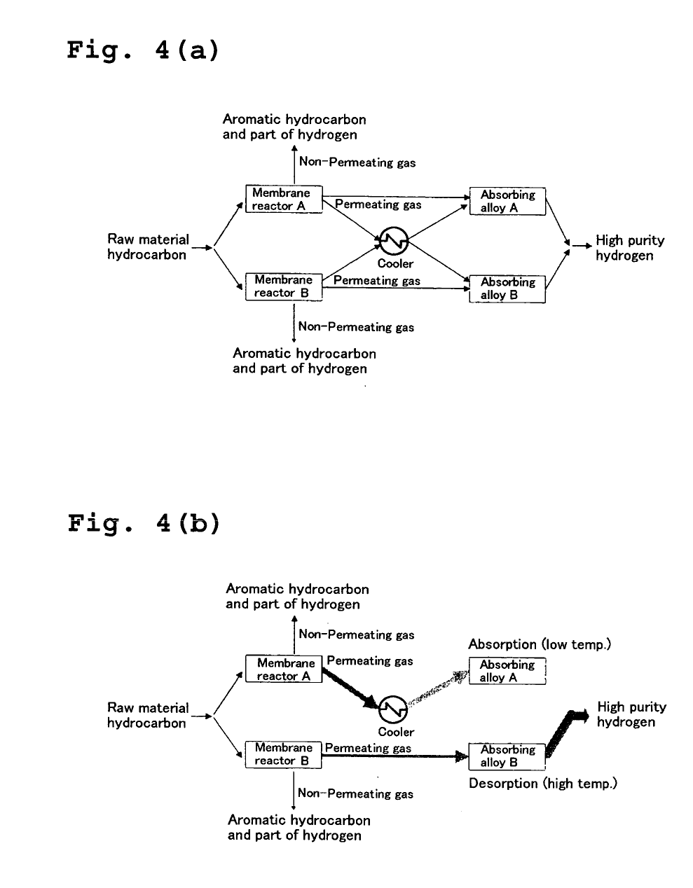

When the reaction condition of the present invention using the membrane reactor is adapted, a hydrogen recovery rate in membrane separation process of not less than 80% can be attained even in adaptation of a reaction tube of simple catalyst filling structure shown in FIG. 3, or a reaction tube in which a catalyst supported by a known granular or pellet-like carrier is filled in a simple double-tube clearance, as well as in adaptation of the inner fin type of FIG. 1 as the outer tube.

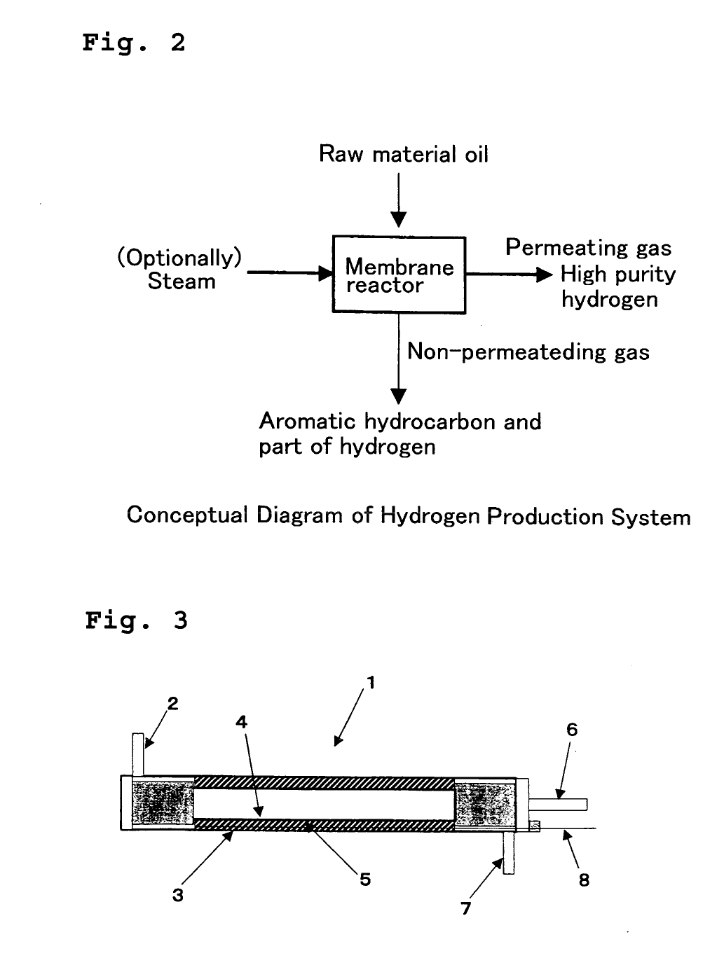

The conceptual diagram of a hydrogen production system of the present invention as the first aspect according to the above is shown in FIG. 2. In the drawing, raw material oil, preferably, hydrocarbon containing cyclohexane ring as a reaction substrate is introduced to a membrane reactor, and converted to hydrogen and unsaturated hydrocarbon such as aromatic hydrocarbon on a dehydrogenation catalyst. The produced hydrogen is membrane-separated through the hydrogen separating membrane, and most of the hydrogen is separated out of the system as permeated gas and made into product hydrogen as high purity hydrogen. A part of the remaining hydrogen and the unsaturated hydrocarbon such as aromatic hydrocarbon are recovered as non-permeated gas. It is preferable from the point of membrane separating operation to introduce, as occasion demands, steam to the permeating side of the hydrogen separating membrane to reduce the hydrogen partial pressure on the permeating side.

The present invention as the second aspect will be then described in detail. Although descriptions are made regardless of duplication with the invention of the first aspect, parts to be omitted will be appropriately pointed out.

In the present invention as the second aspect, also, a so-called membrane reactor is used as the reactor used in a flow reaction system. More accurately, the membrane reactor referred to in the invention as the second aspect is a flow type reaction tube, comprising a dehydrogenation catalyst and a hydrogen separating membrane provided within the reaction tube. In general, the reactor frequently has a double-tube structure in which the hydrogen separating membrane constitutes an inner tube of the reaction tube, and the catalyst is present between an outer tube and the inner tube.

Production of hydrogen by dehydrogenating hydrocarbon using such a reaction tube is performed as follows.

Namely, raw material hydrocarbon is supplied from one side of the flow type reaction tube and fed therein with the clearance of the double tube as a reaction field of dehydrogenation, and dehydrogenated by the catalyst present in the clearance to produce hydrogen and reaction product (including dehydrogenated hydrocarbon, a side reaction product and unreacted hydrocarbon), and the produced hydrogen is simultaneously selectively permeated into the inner tube of the double tube through the hydrogen separating membrane constituting the inner tube, and discharged out of the system, whereby high purity hydrogen is obtained.

According to this method, the produced hydrogen can be rapidly separated, and the dehydrogenation reaction easily proceeds since heat is easily supplied to the double tube clearance where the dehydrogenation reaction that is an endothermic reaction is executed by providing a proper heating means from the outside of the tube. As the proper heating means from the outside of the tube, a known method such as heating by heating medium can be appropriately adapted.

As the hydrogen separating membrane in the second aspect, although a known hydrogen separating membrane having the function capable of selectively separating hydrogen from a mixed gas of hydrocarbon and hydrogen can be used, a metal membrane or a porous inorganic membrane is preferred. Concretely, the hydrogen separating membrane in the first aspect or the like can be used.

The dehydrogenation reaction is carried out using a catalyst. As the catalyst, a solid catalyst having dehydrogenating activity is preferred. As the solid catalyst, a catalyst having dehydrogenating active main component supported by a carrier is suitably usable.

The carrier is preferably composed of a stable metal oxide, or comprises a metal oxide formed on the surface of a support with high heat conductivity (heat conductive support) from the point of high mechanical strength, thermal stability and large surface area.

As the concrete metal oxide and the heat conductive support, those described in the first aspect can be used.

In the second aspect, the dehydrogenation catalyst need not be always arranged in contact with the hydrogen separating membrane, and what is of paramount importance is that the hydrogen produced by the effect of catalyst is immediately and simultaneously subjected to hydrogen membrane separating operation in situ to selectively discharge the hydrogen out of the reaction system of dehydrogenation.

The raw material of dehydrogenation in the present invention is preferably hydrocarbon, further preferably, hydrocarbon having cyclohexane ring. Concretely, those shown in the first aspect can be used.

The reaction condition of dehydrogenation can be properly selected according to the kind of raw material. Concretely, the reaction condition described in the first aspect can be exemplified.

Although the hydrogen produced by dehydrogenation reaction is in a mixed state with the reaction product such as aromatic hydrocarbon, it is immediately subjected to the membrane separating operation using hydrogen separating membrane in situ in the second aspect of the present invention. Similarly to the first aspect, in the membrane reactor that is a flow type reactor used in the second aspect, the side of the hydrogen separating membrane to which hydrogen is separated and permeated is referred to as permeating side, and the opposite side to as non-permeating side. When the flow type reaction tube is made into a double tube with the inner tube composed of a hydrogen separating membrane, operation is performed with the innermost part of the double tube as the hydrogen permeating side and with the clearance of the double tube as the non-permeating side.

One embodiment of the flow type membrane reactor used for the present invention in the second aspect is the one of FIG. 3 described in the first aspect, and this is used in examples and comparative example in the second aspect.

In FIG. 3, a reaction tube 1 is a membrane reactor of double-tube structure, in which an outer tube 3 is composed of a material with high heat conductivity, for example, a metal or the like, and the tube wall of an inner tube 4 is composed of a hydrogen separating membrane (hydrogen permeating membrane 4). An appropriate heating means not shown such as heating by a heat medium is provided on the outside of the double tube. A catalyst 5, which is supported by, for example, a granular appropriate carrier, is filled in the clearance of the double tube, or in the clearance between the outer tube and the inner tube. Raw material gas is introduced into the clearance through a raw material feed pipe 2 located at one end of the double tube, and reaction product and a part of hydrogen are discharged as non-permeated gas through a non-permeated component discharge pipe 7 located at the other end of the clearance. The temperature of dehydrogenation is measured and adjusted by inserting a thermocouple 8 into the clearance. Hydrogen which is selectively membrane-separated is carried in the inner tube 4 as permeated gas. High purity hydrogen is taken out as permeated gas through a permeated gas discharge pipe 6 at the other end of the inner tube 4.

In the present invention in the second aspect, hydrogen absorbing (storing) alloy is connected to the permeating side to absorb at least part of the resulting hydrogen to the alloy, whereby the internal pressure of the permeating-side system is reduced lower than that of the non-permeating side system (reaction field) to improve the hydrogen recovery rate.

Namely, the hydrogen gas obtained from the permeating side is cooled by a heat exchanger, and then introduced to the hydrogen absorbing (storing) alloy. The pressure in the permeating-side system is reduced lower than that in the non-permeating side system (corresponding to the above-mentioned reaction pressure) by use of the hydrogen absorbing capability at low temperature of the hydrogen absorbing (storing) alloy.

In the present invention as the second aspect, although a permeating-side pressure lower than that in the non-permeating side system (corresponding to the above-mentioned reaction pressure) is sufficient, the pressure is set preferably to not more than 0.1 MPa, further preferably to not more than 0.05 MPa.

The hydrogen absorbing (storing) alloy in the present invention as the second aspect means a composite composed of at least one metal component and a nonmetallic component such as other metals or halogen which can absorb hydrogen in the form of metal hydride or the like, and has the reversibility of desorbing hydrogen when heated and absorbing hydrogen when cooled. As the hydrogen absorbing (storing) alloy to be used in the present invention, any alloy having hydrogen absorbing capability, including known hydrogen absorbing (storing) alloys, can be used without having a particular limit to the kind.

Examples of the hydrogen absorbing (storing) alloy include (1) alloy of Mg or Ca (in this case, Ni, Cu, Ti or the like is used as the counter component, and concrete examples of the alloy include Mg₂Ni, Mg₂Cu, TiCu, LaMg, and CaNi); (2) alloy of Ti, Zr, V or Nb (Fe or the like is used as the counter component, and concrete examples of the alloy include FeTi-based alloy, Be₂Ti, Be₂Zr, ZrX (X=halogen), NiZr, TiCu, TiCrFe, TiZrCeFeMnCu, and ZrTiCrFeMnCu); (3) rare earth alloy represented by LaNi₅-based alloy or an alloy with La substituted by Millish metal (Mm) (wherein Millish metal is a cerium rare earth element mixture of 40-50 mass % of Ce, 20-40 mass % of La, and the like, and concrete examples of the alloy include LaNi₅, MmNi₅, MmCaNiAl, and CaMmNiAl); (4) Pd-based alloy (concrete examples thereof include amorphous alloy such as Pd₈₃Si₁₇ or Pd₃₅Zr₆₅); and mixtures of two or more kinds thereof.

Among them, particularly, alloys which can contain (absorb) not less than 1 mass % of hydrogen are preferred. Of course, the most preferable alloy which has the reversibility of desorbing the absorbed hydrogen by temperature change or the like is the alloy described in the above (2).

The shape of the hydrogen absorbing (storing) alloy is not particularly limited, but a granular shape is preferred. As the grain size, a diameter of about 0.1 to 10 mm is preferred.

Conventional examples describing improvements or usage of these hydrogen absorbing bodies include Japanese Patent Application Laid-Open Nos. 61-132501, 61-233516, 3-184275, and 4-22063, and the like.

Since the hydrogen absorbing (storing) alloy has the property of desorbing hydrogen when heated and absorbing (storing) hydrogen when cooled, the hydrogen absorbing (storing) alloy is further preferably filled in a temperature-controllable container.

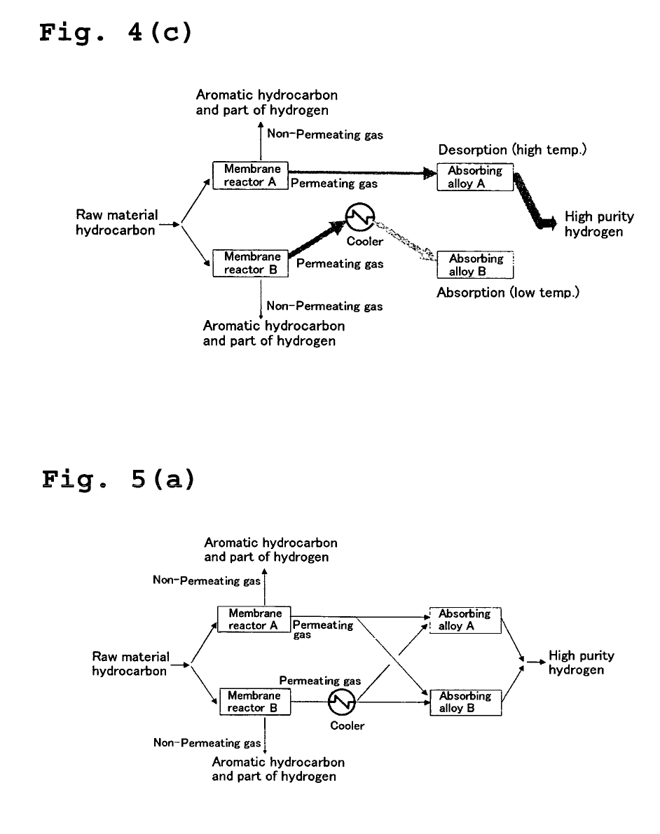

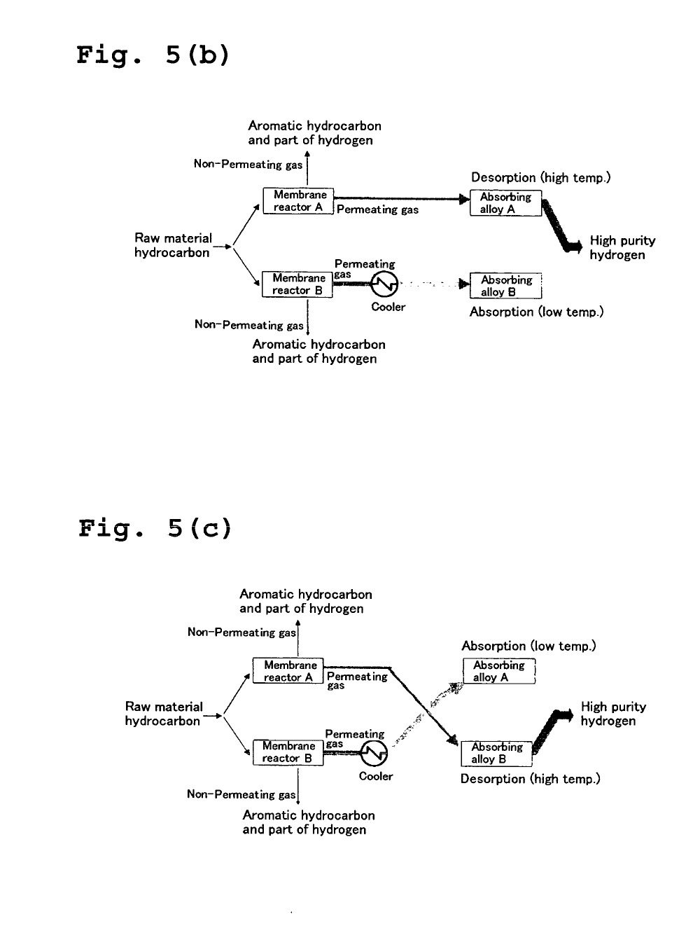

Conceptual diagrams of the hydrogen production system used for the method for producing hydrogen of the present invention are shown in FIGS. 4 and 5.

As shown in FIG. 4(a), the system comprises two membrane reactors A and B, two hydrogen absorbing (storing) alloy units A and B having hydrogen absorbing (storing) alloys in containers (absorbing (storing) alloys A and B in the drawing), a cooler, and piping for connecting them.

Raw material hydrocarbon, preferably, hydrocarbon containing cyclohexane ring is introduced into the membrane reactors, and converted to hydrogen and unsaturated hydrocarbon such as aromatic hydrocarbon on a dehydrogenation catalyst. Most of the produced hydrogen is separated in situ as permeated gas through the hydrogen separating membrane, and part of the remaining hydrogen and the unsaturated hydrocarbon such as aromatic hydrocarbon are recovered as non-permeated gas. The hydrogen on the permeating-side is cooled by the cooler and absorbed to the hydrogen absorbing (storing) alloy, or produces hydrogen with the hydrogen desorbed from the hydrogen absorbing (storing) alloy while bypassing the cooler. This process is periodically repeated.

As shown in the period 1 of FIG. 4 (b), the permeated gas of the membrane reactor A is cooled by the cooler and absorbed to the absorbing (storing) alloy A having a closed outlet. At that time, in the membrane reactor A, the pressure in the permeating-side system is reduced, compared with that in the non-permeating side system, and the hydrogen recovery rate is improved. The permeated gas of the membrane reactor B is introduced to the absorbing (storing) alloy B while keeping high temperature after reaction. According to this, the absorbing (storing) alloy B is heated to desorb the hydrogen absorbed in the previous period, which produces hydrogen with the hydrogen produced in the membrane reactor B.

As shown in the period 2 of FIG. 4(c), the permeated gas of the membrane reactor B is cooled by the cooler and absorbed to the absorbing (storing) alloy B having a closed outlet. At that time, in the membrane reactor B, the pressure in the permeating-side system is reduced, compared with in the non-permeating-side system, and the hydrogen recovery rate is improved. The permeated gas of the membrane reactor A is introduced to the absorbing (storing) alloy while keeping high temperature after reaction. According to this, the absorbing (storing) alloy A is heated to desorb the hydrogen absorbed in the period 1, which produces hydrogen with the hydrogen produced in the membrane reactor A.

Another example of the system of the present invention is shown in FIG. 5. As shown in FIG. 5(a), the system comprises two membrane reactors (A, B), two hydrogen absorbing (storing) alloy units having hydrogen absorbing (storing) alloys put in containers (absorbing (storing) alloys A, B in the drawing), a cooler and piping for connecting them.

Although the conception is basically the same as that in FIG. 4, the piping structure is differed from that in FIG. 4, wherein the permeated hydrogen of the membrane reactor B is regularly absorbed to the hydrogen absorbing (storing) alloy A or B, and the permeated hydrogen of the membrane reactor A regularly produces hydrogen with the desorbed hydrogen in the hydrogen absorbing (storing) alloy A or B.

This system is periodically operated as shown in FIGS. 5 (b) and (c).

As shown in the period 1 of FIG. 5(b), the permeated gas of the membrane reactor B is cooled by the cooler and absorbed to the absorbing (storing) alloy B having a closed outlet. At that time, in the membrane reactor B, the pressure in the permeating-side system is reduced, compared with that in the non-permeating-side system, and the hydrogen recovery rate is improved. The permeated gas of the membrane reactor A is introduced to the absorbing (storing) alloy A while keeping high temperature after reaction. According to this, the absorbing (storing) alloy A is heated to desorb the hydrogen absorbed in the previous period, which produces hydrogen with the hydrogen produced in the membrane reactor A.

As shown in the period 2 of FIG. 5(c), the permeated gas of the membrane reactor B is cooled by the cooler and absorbed to the absorbing (storing) alloy A having a closed outlet. At that time, in the membrane reactor B, the pressure in the permeating-side system is reduced, compared with that in the non-permeating-side system, and the hydrogen recovery rate is improved. The permeated gas of the membrane reactor A is introduced to the absorbing (storing) alloy B while keeping high temperature after reaction. According to this, the absorbing (storing) alloy B is heated to desorb the hydrogen absorbed in the period 1, which produces hydrogen with the hydrogen produced in the membrane reactor A.

In each system shown in the drawings, the switching of period is performed before the hydrogen absorbing (storing) alloys are saturated. Although the membrane reactors and the hydrogen absorbing (storing) alloy units are arranged by twos in the drawings, it is preferable to arrange them by twos or more from the point of further continuous production of hydrogen.

### EXAMPLES

The present invention will be further described in detail according to experimental examples and working examples. The present invention is never limited to the range of these experimental examples and working examples.

### [Separating Membrane]

A ceramic membrane having a hydrogen transmission coefficient of 4.2×10⁷ mol/m²/sec/Pa and a toluene transmission coefficient of 2.8×10¹¹ mol/m²/sec/Pa was used as a separating membrane A, the ceramic membrane comprising one α-alumina layer formed on the inner surface of a porous ceramic tube-shaped support having an outer diameter of 10 mm, an inside diameter of 8.4 mm and a length of 300 mm, three γ-alumina layers formed thereon, one silica layer formed thereon, and a silica thin film formed on the outermost surface.

A palladium film having a hydrogen transmission coefficient of 200 cc/cm²/min/atm^{1/2} and a film thickness of 2.5 μm is used as a separating membrane B, the film comprising palladium and silver (Pd:Ag=85:15) applied to the outer surface of a porous ceramic tubular support having an outer diameter of 10 mm, an inside diameter of 8.4 mm and a length of 300 mm by electroless plating.

### [Catalyst]

A spherical commercially available catalyst with an average diameter of 1.5 mm in which 0.3 mass % of platinum is supported by a γ-alumina carrier is used as catalyst A.

The fin part of an aluminum-made internal fin type reaction tube shown in FIG. 1 with an inside diameter of 24 mm, a length of 300 mm, and a fin length of 6 mm is washed with diluted nitric acid, washed with water, and dried, and anode oxidation is performed thereto in a chromic acid aqueous solution. After the operation of applying a commercially available pseudo boehmite sol to the fin part followed by drying is repeated four times, baking was performed at 450° C. for 2 hours, and 0.2504 g of platinum is supported in the fin part by impregnation using an aqueous solution of chloroplatinic acid. Thereafter, baking is preformed at 300° C. for 2 hours, whereby catalyst B is obtained.

### Example 1

In the clearance between a reaction tube with an inside diameter of 24 mm and a length of 300 mm and a hydrogen separating membrane in a membrane reactor schematically shown in FIG. 3, 110 cc of catalyst A was filled, methylcyclohexane was introduced as raw material, and dehydrogenation reaction was performed in a condition with reaction pressure 0.5 MPa (absolute pressure), permeating-side pressure 0.1 MPa (absolute pressure), reaction temperature 300° C., and LHSV 0.5, 1.0, 2.0 h⁻¹. The results using the separating membrane A and the separating membrane B as the hydrogen separating membrane are shown in Table 1. The LHSV is a flow velocity, which is represented by “methylcyclohexane liquid volume (cc)/catalyst (cc)/time (h)”. The hydrogen recovery rate is represented by “hydrogen (mol) recovered from the membrane reactor permeating side/theoretical hydrogen generation quantity (mol) from methylcyclohexane introduced×100”.

### Example 2

Hydrogen was produced in the same manner as Example 1, except setting the catalyst B as a reaction tube in the membrane reactor instead of the catalyst A. The result is shown in Table 1.

### Example 3

As the catalyst 5, 110 cc of the catalyst A was filled in the clearance between the reaction tube 3 with an inside diameter of 24 mm and a length of 300 mm and the hydrogen separating membrane (hydrogen permeating membrane 4) in the membrane reactor schematically shown in FIG. 3, methylcyclohexane was introduced thereto as raw material gas, and dehydrogenation reaction was carried out in a condition with reaction pressure 0.2, 0.4, 0.6, 0.8 MPa (absolute pressure), permeated gas-side pressure 0.1 MPa (absolute pressure), reaction temperature (catalyst layer outlet temperature) 300, 270, 240° C., and LHSV 0.5 h⁻¹. The result using the separating membrane B as the hydrogen separating membrane (hydrogen permeating membrane 4) is shown in Table 2.

### Example 4

Hydrogen was produced in the same manner as Example 3, except setting the catalyst B as the reaction tube in the membrane reactor instead of the catalyst A. The result is shown in Table 3.

### Example 5

Hydrogen was produced in the same manner as Example 4, except introducing steam to the permeating side of the hydrogen separating membrane and performing the reaction with a permeating-side hydrogen partial pressure of 0.05 MPa. The result is shown in Table 4.

### Example 6

Hydrogen was produced in the same manner as Example 4, except introducing steam to the permeating side of the hydrogen separating membrane and performing the reaction with a permeating-side hydrogen partial pressure of 0.01 MPa in a condition with reaction temperature (catalyst layer outlet temperature) 300, 270, 240, 220° C. The result is shown in Table 5.

As shown in Table 1, the hydrogen recovery rate was reduced as the LHSV of the raw material is increased from 0.5 to 2.0 h⁻¹ in Example 1 using the catalyst A, while the hydrogen recovery rate was substantially unchanged in the LHSV range from 0.5 to 2.0 h⁻¹ in Examples 2 and 4 using the catalyst B excellent in heat conductivity. This shows that the catalyst B has significantly higher performance than the catalyst A. Both the hydrogen permeating membranes A and B exhibit sufficient performance in this reaction condition.

As shown in Tables 2 and 3, at a permeating-side pressure of 0.1 MPa, a hydrogen recovery rate of not less than 80% can be realized in an area with a reaction temperature of not lower than 300° C. and a reaction pressure of not less than 0.4 MPa. The range capable of realizing the hydrogen recovery rate of not less than 80% is the same also in the case using the catalyst B higher in performance than the catalyst A.

Further, as shown in Table 4, in the condition with permeating-side hydrogen partial pressure of 0.05 MPa by steam introduction, hydrogen recovery rate of not less than 80% can be realized in an area with a reaction temperature of not lower than 270° C. and a reaction pressure of not less than 0.4 MPa. As shown in Table 5, also, in the condition of a permeating-side hydrogen partial pressure of 0.01 MPa by steam introduction, the hydrogen recovery rate of not less than 80% can be realized in an area with a reaction temperature of not lower than 220° C. and a reaction pressure of not less than 0.2 MPa.

Examples of the second aspect will be described, wherein the device having the structure described below was used for examples and comparative example of the second aspect.

### [Separating Membrane]

A ceramic membrane having a hydrogen transmission coefficient of 4.2×10⁻⁷ mol/m²/sec/Pa and a toluene transmission coefficient of 2.8×10⁻¹⁰ mol/m²/sec/Pa was used as a separating membrane A, the ceramic membrane comprising one α-alumina layer formed on the inner surface of a porous ceramic tubular support having an outer diameter of 10 mm, an inside diameter of 8.4 mm and a length of 300 mm, three γ-alumina layers formed thereon, one silica layer formed thereon, and a silica thin film formed on the outermost surface.

A palladium membrane having a hydrogen transmission coefficient of 200 cc/cm²/min/atm^{1/2} and a film thickness of 2.5 μm was used as a separating membrane B, the membrane comprising palladium and silver (Pd:Ag=85:15) applied to the outer surface of a porous ceramic tubular support having an outer diameter of 10 mm, an inside diameter of 8.4 mm and a length of 300 mm by electroless plating.

### [Catalyst]

A spherical commercially available catalyst with an average diameter of 1.5 mm in which 0.3 mass % of platinum is supported by a γ-alumina carrier is used.

### [Membrane Reactor]

The membrane reactor having the structure shown in FIG. 3 was used. The reaction tube has an inside diameter of 24 mm and a length of 300 mm. The separating membrane A (in Example 1 of the second aspect) or the separating membrane B (in Example 2 and Comparative Example of the second aspect) was provided in the inner part thereof. In the clearance between the reaction tube and the hydrogen separating membrane, 110 cc of the catalyst was filled.

[Hydrogen Absorbing (Storing) Alloy]

Filled 500 g of hydrogen absorbing (storing) alloy Ti_{0.6}Zr_{0.4}Mn_{0.8}CrCu_{0.2} in a container, was used.

### [System]

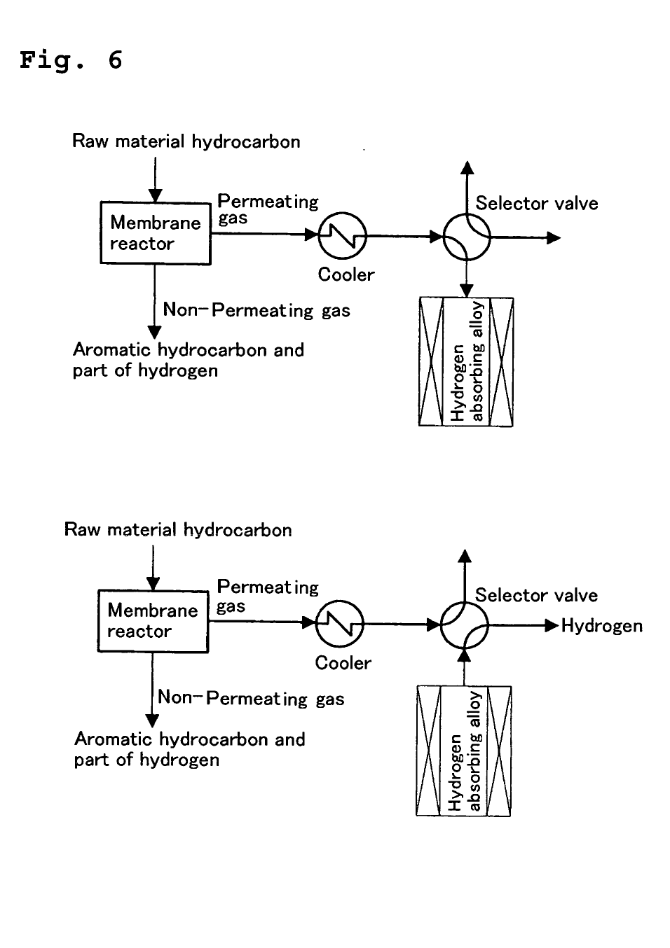

As schematically shown in FIG. 6, the membrane reactor, a cooler, the hydrogen absorbing (storing) alloy, and a selector valve are connected through piping.

### [Comparative Example of Second Aspect]

The separating membrane B was used in the membrane reactor. The cooler, the hydrogen absorbing (storing) alloy and the selector valve are not used. Methylcyclohexane was introduced as raw material to the catalyst layer, and dehydrogenation reaction was performed in a condition with reaction pressure 0.2, 0.4, 0.6, 0.8 MPa (absolute pressure), permeating-side pressure 0.1 MPa (absolute pressure), reaction temperature (catalyst layer outlet temperature) 300, 280, 260° C., and LHSV 0.5 h⁻¹. The result is shown in Table 6.

The LHSV (liquid hourly space velocity) is represented by “methylcyclohexane liquid volume (cc)/catalyst (cc)/time (h)”. The hydrogen recovery rate is represented by “hydrogen (mol) recovered from the membrane reactor permeating side/theoretical hydrogen generation quantity (mol) from methylcyclohexane introduced×100”.

### (Example 1 of Second Aspect)

The device shown in FIG. 6 was used. The reaction was performed for 5 minutes in the same manner as in Comparative Example, as shown in FIG. 6(a), in a state where hydrogen produced in the membrane reactor is absorbed to the hydrogen absorbing (storing) alloy. The permeated gas was cooled during hydrogen absorption so that the permeating-side hydrogen pressure was below 0.007 MPa.

The selector valve was switched as shown in FIG. 6(b), hydrogen is desorbed by heating the hydrogen absorbing (storing) alloy to 170° C., and the quantity of hydrogen produced for 5 minutes in the state of FIG. 6(a) was measured.

The result is shown in Table 6.

### (Example 2 of Second Aspect)

The same experiment as in Example 1 of the second aspect was performed, except using the separating membrane A as the hydrogen separating membrane. The result is shown in Table 6.

As shown in Table 6, the hydrogen recovery rate can be improved in Examples 1 and 2 of the second aspect where the permeating-side pressure is reduced by the hydrogen absorption of hydrogen absorbing (storing) alloy, in contrast to Comparative Example with a permeating-side pressure of 0.1 MPa.

### INDUSTRIAL APPLICABILITY

Since the hydrogen obtained by the method for producing hydrogen according to the present invention has high purity, it can be used as various chemical materials and raw materials, including fuel cell hydrogen. Particularly, this method is suitable as a production method of fuel cell hydrogen because of its small scale and easiness.

1. A method for producing hydrogen, comprising:
providing a hydrogen removing means using a hydrogen separating membrane within a dehydrogenation reaction system adapted to dehydrogenate hydrocarbon having cyclohexane ring in a flow type reaction tube containing a catalyst supported by a carrier composed of a metallic heat conductive support having a metal oxide layer localized on the surface thereof to produce hydrogen and aromatic hydrocarbon; and
performing membrane separating operation through said hydrogen separating membrane while conducting said dehydrogenation reaction to remove mainly the hydrogen to the permeating side thereof and obtain mainly the aromatic hydrocarbon on the non-permeating side thereof.

2. The method for producing hydrogen according to claim 1, wherein said hydrocarbon having cyclohexane ring includes methylcyclohexane, and toluene produced by the dehydrogenation thereof is separated.

3. The method for producing hydrogen according to claim 2, wherein said hydrogen separating membrane is a ceramic membrane.

4. The method for producing hydrogen according to claim 2, wherein said hydrogen separating membrane is a metallic membrane containing 100 to 10 mass % of Pd.

5. The method for producing hydrogen according to claim 2, wherein said carrier in said catalyst is a carrier containing alumina.

6. The method for producing hydrogen according to claim 2, wherein said reaction tube has a double-tube structure with an outer tube composed of a metallic heat conductive support body and an inner tube composed of a hydrogen separating membrane, a plurality of metallic heat conductive fin-like projections extending inwardly from the outer tube are provided long in a flowing direction in the clearance of said double tube, and a metal oxide layer is localized on at least the fin-like projection surface to support the catalyst.

7. A hydrogen production reaction tube for simultaneously performing dehydrogenation of hydrocarbon and membrane separation of resulting hydrogen, which is a double-tube flow type reaction tube with an outer tube composed of a metallic heat conductive support and an inner tube composed of a hydrogen separating membrane, comprising a plurality of metallic heat conductive fin-like projections extending inwardly from the outer tube, which are provided long in a flowing direction in the clearance of the double tube, and a metal oxide layer localized on at least the fin-like projection surface to support a catalyst.

8. A method for producing hydrogen, comprising continuously permeating and separating, within a flow type reaction system provided with a dehydrogenation catalyst and a hydrogen separating membrane, produced hydrogen through said hydrogen separating membrane while dehydrogenating hydrocarbon therein; and absorbing at least part of the resulting hydrogen flow to a hydrogen absorbing (storing) alloy to make the pressure on the hydrogen permeating side of said hydrogen separating membrane lower than that on the non-permeating side thereof.

9. The method for producing hydrogen according to claim 8, wherein said hydrocarbon includes hydrocarbon having cyclohexane ring.

10. The method for producing hydrogen according to claim 9, wherein said hydrocarbon having cyclohexane ring is methylcyclohexane.

11. The method for producing hydrogen according to claim 9, wherein said hydrogen separating membrane is a ceramic membrane.

12. The method for producing hydrogen according to claim 9, wherein said hydrogen separating membrane is a metallic membrane containing 100 to 10 mass % of Pd.

13. A hydrogen production system comprising:
two or more flow type membrane reactors,
two or more hydrogen absorbing alloy units,
a cooler,
passages for connecting them, and a passage switching means, and being adapted to perform absorption and desorption of hydrogen in the hydrogen absorbing alloy units by periodically switching the passages.

14. The hydrogen production system according to claim 13,
wherein said passage switching means periodically switches a flow for cooling a permeated hydrogen flow from one flow type membrane reactor by the cooler to absorb it to one hydrogen absorbing (storing) alloy unit and a flow for supplying a permeated hydrogen flow from the other flow type membrane reactor to the hydrogen absorbing alloy unit which absorbed hydrogen without through the cooler, whereby hydrogen from said flow type membrane reactor and desorbed hydrogen from said hydrogen absorbing alloy are obtained.

15. The method for producing hydrogen according to claim 8, wherein said hydrogen separating membrane is a ceramic membrane.

16. The method for producing hydrogen according to claim 8, wherein said hydrogen separating membrane is a metallic membrane containing 100 to 10 mass % of Pd.

17. The method for producing hydrogen according to claim 10, wherein said hydrogen separating membrane is a ceramic membrane.

18. The method for producing hydrogen according to claim 10, wherein said hydrogen separating membrane is a metallic membrane containing 100 to 10 mass % of Pd.

19. The method for producing hydrogen according to claim 1, wherein said hydrogen separating membrane is a ceramic membrane.

20. The method for producing hydrogen according to claim 1, wherein said hydrogen separating membrane is a metallic membrane containing 100 to 10 mass % of Pd.

21. The method for producing hydrogen according to claim 1, wherein said carrier in said catalyst is a carrier containing alumina.

22. The method for producing hydrogen according to claim 1, wherein said reaction tube has a double-tube structure with an outer tube composed of a metallic heat conductive support body and an inner tube composed of a hydrogen separating membrane, a plurality of metallic heat conductive fin-like projections extending inwardly from the outer tube are provided long in a flowing direction in the clearance of said double tube, and a metal oxide layer is localized on at least the fin-like projection surface to support the catalyst.

23. The method for producing hydrogen according to claim 3, wherein said hydrogen separating membrane is a metallic membrane containing 100 to 10 mass % of Pd.

24. The method for producing hydrogen according to claim 3, wherein said carrier in said catalyst is a carrier containing alumina.

25. The method for producing hydrogen according to claim 3, wherein said reaction tube has a double-tube structure with an outer tube composed of a metallic heat conductive support body and an inner tube composed of a hydrogen separating membrane, a plurality of metallic heat conductive fin-like projections extending inwardly from the outer tube are provided long in a flowing direction in the clearance of said double tube, and a metal oxide layer is localized on at least the fin-like projection surface to support the catalyst.

26. The method for producing hydrogen according to claim 4, wherein said carrier in said catalyst is a carrier containing alumina.

27. The method for producing hydrogen according to claim 4, wherein said reaction tube has a double-tube structure with an outer tube composed of a metallic heat conductive support body and an inner tube composed of a hydrogen separating membrane, a plurality of metallic heat conductive fin-like projections extending inwardly from the outer tube are provided long in a flowing direction in the clearance of said double tube, and a metal oxide layer is localized on at least the fin-like projection surface to support the catalyst.
